(19) United States
(12) Patent Application Publication
(10) Pub. No.: US 2018/0127837 A1
(43) Pub. Date: May 10, 2018

(54) CONSTRUCTION AND APPLICATION OF ONE INNOVATIVE EXPRESSION VECTOR FOR VIRUS-LIKE PARTICLES

(71) Applicant: Chinese Academy of Inspection and Quarantine, Beijing (CN)

(72) Inventors: Junhua Deng, Beijing (CN); Shaoqiang Wu, Beijing (CN); Xiangmei Lin, Beijing (CN); Yongning Zhang, Beijing (CN); Jizhou Lv, Beijing (CN); Chunyan Feng, Beijing (CN); Caixia Wang, Beijing (CN)

(73) Assignee: Chinese Academy of Inspection and Quarantine, Beijing (CN)

(21) Appl. No.: 15/329,881

(22) PCT Filed: Dec. 22, 2015
(86) PCT No.: PCT/CN2015/098298

§ 371 Date: Jan. 27, 2017

(51) Int. Cl. C12Q 1/70; C12N 15/66; C12N 7/00

(52) U.S. Cl. CPC C12Q 1/70; C12N 15/66; C12N 7/00; C12Q 2600/166; C12N 2760/00023; C12N 2740/00051; C12N 2760/00051; C12N 2740/00043

(57) ABSTRACT

The present invention provides a novel virus-like particle expression vector pTMSCA2C, which is constructed as follows: firstly, using plasmid pTrcHis-MS2 as a starting vector and mutating the base T at position 5 of the gene sequence of MS2 bacteriophage 19mer packaging site on the plasmid pTrcHis-MS2 into C through genetic mutation technologies to obtain a plasmid pTMSC; then, mutating valine which is a amino acid corresponding to the initiation codon on the plasmid pTMSC for encoding the maturase protein of MS2 bacteriophage into methionine to obtain a plasmid pTMSCA; and finally, the gene sequence coding wild type MS2 bacteriophage coat protein, after the removal of the terminator, is linked in series with the gene sequence coding MS2 bacteriophage coat protein comprising histidine-tag which is from a pseudovirus vector pTrcMS, and the gene sequence obtained after linking in series is linked to the plasmid pTMSCA to give pTMSCA2C. When the virus-like particle is prepared by using the expression vector pTMSCA2C of the present invention, the yield and purity of the virus-like particle may be improved while the workload for preparation of virus-like particles may be greatly reduced.

### TECHNICAL FIELD

The present invention relates to the field of genetic engineering, and more particularly, relates to a novel virus-like particle expression vector, construction methods and applications thereof.

### BACKGROUND ART

At the end of the 20^{th} century, Pasloske et al. proposed a new preparation technique of RNA quality control products, i.e., Armored RNA technique. The principle of this technique is that a sequence comprising a coat protein gene of E. coli bacteriophage MS2, and an exogenous gene are cloned into a expression vector, such a vector may transcribe the exogenous gene into RNAs and use the assembled coat protein that are produced by the gene encoding the MS2 coat protein on the vector to assemble the RNAs into RNA-protein complexes having a structure of spherical RNA viruses. The RNA-protein complexes are referred to as Armored RNA virus-like particles.

In traditional Armored RNA preparation techniques, when the exogenous RNA fragments packaged by the virus-like particles (Virus-Like-Particles, VLPs) expression vector are generally greater than 500 bp, the packaging efficiency is significantly reduced. For the design of a virus-like particle expression vector, improving the ability of packaging the length of the exogenous RNA may make the vector have more extensive application value in the quality control and standardization of clinical detection of RNA pathogenic microorganisms. For example, the target genes of different pathogenic microorganisms may be constructed together to form a chimera, and packaged into the coat protein to form VLPs. Such VLPs may simultaneously perform detection and quality control of multiple pathogenic microorganisms, save the cost and simplify the operation procedures. Additionally, with respect to the detection of a specific pathogenic microorganism, the amplified target fragments of RT-PCR kits produced by different manufacturers may vary. If those different target fragments are constructed into the same RNA chain and packaged into the MS2 coat protein to form VLPs, direct comparison of results between different laboratories may be achieved.

Depending on the difference of affinity and the number of packaging sites, the length of exogenous fragments packaged by the virus-like particle expression vector may vary. \textcircled{1} The types of the packaging sites are different. For the wild type MS2 bacteriophage 19mer, if the exogenous fragments are greater than 500 bp, the packaging efficiency will decrease gradually, the amount of the expressed virus-like particles will decrease greatly and thus the purified virus-like particles will decrease correspondingly. Some of the sites of MS2 19mer may be modified by genetic mutation means, which will greatly increase the length of exogenous fragments packaged by the vector. Only nucleotides at 4 positions in the stem-loop structure (a hairpin structure) of 19mer of MS2 RNA play a particularly important role in the recognition of the coat protein. In the case where the base pairing of the stem is ensured, adenine at positions 4, 7 and 10 and pyrimidine at position 5 may interact with the envelope protein. \textcircled{2} The numbers of the packaging sites are different. For the same expression vector, introducing an exogenous fragment into the vector while increasing packaging sites may greatly increase the length of the packaged fragment and the packaging efficiency. Meanwhile, the expression vectors have different promoters and are transcribed into RNA to different extent. Qiuying et al. (2006) applied a single plasmid system to express VLPs comprising the exogenous chimera RNA (1200 bp) of SARS-CoV2 and HCV by using pSE380 as a vector and removing unnecessary multiple cloning sites on the pAR-1 vector. WEI Yuxiang et al. (2008) realized the expression of VLPs comprising the exogenous chimera RNA (1891 bp) of H5N1, HCV and SARS-CoV by using pET-28 (b) as a vector and increasing MS2 packaging sites. However, the above-mentioned studies merely solve the problem of the length of the packaged exogenous fragment to some extent, but do not change the complex preparation processes of virus-like particles.

ZL201110445022.5 discloses a pseudovirus vector pTrcMS, and specifically discloses that the virus-like particles are obtained by column purification of protein through 6×His purification tag added between 15^{th} and 16^{th} amino acids of the coat protein of MS2 bacteriophage, which greatly simplifies the complex operation procedure of preparing the virus-like particles and increases the quality of the purified virus-like particles. However, the yield of the virus-like particles prepared based on the pTrcMS vector would decrease significantly, which limits the application of virus-like particles.

### SUMMARY OF THE INVENTION

One objective of the present invention is to provide a novel virus-like particle expression vector pTMSCA2C.

Another objective of the present invention is to provide a construction method and applications of the novel virus-like particle expression vector pTMSCA2C.

In order to realize the objectives of the present invention, the novel virus-like particle expression vector pTMSCA2C of the present invention is constructed as follows: using plasmid pTrcHis-MS2 as a starting vector and mutating the base T at position 5 of the gene sequence of MS2 bacteriophage 19mer packaging site on the plasmid pTrcHis-MS2 into C through genetic mutation technologies to obtain a plasmid pTMSC; then mutating valine which is a amino acid corresponding to the initiation codon on the plasmid pTMSC for encoding the maturase protein of MS2 bacteriophage into methionine to obtain a plasmid pTMSCA; and finally, the gene sequence coding wild type MS2 bacteriophage coat protein, after the removal of the terminator, is linked in series with the gene sequence coding MS2 bacteriophage coat protein comprising histidine-tag (6×His protein purification tag) which is from the pseudovirus vector pTrcMS, and the gene sequence obtained after linking in series which encodes a double-CP protein structure is linked to the plasmid pTMSCA to give pTMSCA2C. Wherein, the nucleotide sequence of the plasmid pTrcHis-MS2 (ZL201110445022.5) is shown in SEQ ID NO: 1, and the nucleotide sequence of the pseudovirus vector pTrcMS is shown in SEQ ID NO: 2.

The nucleotide sequence of the novel virus-like particle expression vector pTMSCA2C of the present invention is shown in SEQ ID NO: 3.

The present invention also provides a construction method of said expression vector, comprising the following steps:

1) preparation of a plasmid pTMSC: using a plasmid pTrcHis-MS2 as a template, PCR amplification is performed with Primer A and Primer B into which a base mutation is introduced respectively to obtain PCR product A and PCR product B, and then the amplified products are recovered and purified; the plasmid pTrcHis-MS2 is subjected to double restriction enzyme digestion with XhoI and HindIII and the digested plasmid pTrcHis-MS2 is linked with the PCR product A and PCR product B by In-Fusion technique and transformed into a recipient; and after screening and identification, the plasmid pTMSC is obtained;

2) preparation of a plasmid pTMSCA: using the plasmid pTMSC as a template, PCR amplification is performed with Primer 1 into which a base mutation is introduced to obtain PCR product I, and then the amplified product is recovered and purified; the plasmid pTMSC is subjected to double restriction enzyme digestion with NcoI and PmaCI and the digested plasmid pTMSC is linked with the PCR product I by In-Fusion technique and transformed into a recipient; and after screening and identification, the plasmid pTMSCA is obtained; and

3) construction of a expression vector pTMSCA2C: a gene sequence coding wild type MS2 bacteriophage coat protein, after the removal of terminator, is linked in series with a gene sequence coding MS2 bacteriophage coat protein comprising histidine-tag from a pseudovirus vector pTrcMS, and the gene sequence obtained after linking in series is inserted into the plasmid pTMSCA between XhoI and HindIII restriction enzyme cutting sites to give the expression vector pTMSCA2C.

Wherein, the sequences of the Primer A, Primer B and Primer 1 are as follows:

Primer A-F:
5′-GAGGAATAAACCATGCGAGCTTTTAGTACCCTTG-3′;
Primer A-R:
5′-TGGGTGATCCTCATGTTTGAATGGCCGGCGTC-3′;
Primer B-F:
5′-GCCATTCAAACATGAGGATCACCCATGTCGAAG-3′
Primer B-R:
5′-GTTCGGGCCCAAGCTTCGAATTCCC-3′;
Primer 1-F
5′-GAGGAATAAACCATGCGAGCTTTTAGTACCCTTG-3′;
and
Primer 1-R:
5′-CCACCTGCCGGCCACGTGTTTTGATC-3′.

The primers used for identification in steps 1) and 2) of the above-mentioned methods are:

Primer-U1:
5′-GACAATTAATCATCCGGCTCG-3′,
and
Primer-L1: 5′-GATCTTCGTTTAGGGCAAGGTAG-3′.

The present invention also provides a virus-like particle comprising a RNA transcript of the exogenous genes carried by the the novel virus-like particle expression vector pTMSCA2C.

The present invention also provides a method for the preparation of virus-like particles, comprising the following steps: an exogenous gene fragment is cloned into the downstream of the gene coding sequences of MS2 bacteriophage coat protein linked in series in the novel virus-like particle expression vector pTMSCA2C; then, a terminator is inserted into the downstream of the exogenous gene fragment; after transcription, RNA transcripts of the exogenous gene carrying a RNA sequence of a bacteriophage operator are obtained; bacteriophage coat proteins are expressed after induction and assembled into protein coats while the RNA transcripts of the carried exogenous gene are encapsulated into the protein coats to give the virus-like particles.

The present invention also provides the uses of the novel virus-like particle expression vector or the virus-like particle in the preparation of quality control products for detection of pathogenic microorganisms.

The present invention further provides the quality control products prepared from the virus-like particles used for the detection of pathogenic microorganisms.

Additionally, the present invention further provides the uses of the virus-like particle in the detection of animal pathogenic microorganisms, which is primarily acted as a reference material and applied in the detection technologies for nucleic acid of various animal pathogenic microorganisms.

When the virus-like particle is prepared by using the expression vector pTMSCA2C of the present invention, the yield and purity of the virus-like particle may be improved while the workload for the preparation of the virus-like particles may be greatly reduced. The virus-like particle prepared according to the present invention has at least one of the following advantages:

(i) Increased packing capacity of the exogenous genes. Since the bases of the 19mer stem-loop structure of bacteriophage MS2 genome have been changed, the affinity between the packaging sites and the coat protein is greatly improved and long fragments of the exogenous genes may be packaged into the coat protein successfully to form VLPs.

(ii) Easiness of purification. Since the 6×His protein purification tag, which may be twisted freely, is added to the AB-loop hairpin structure of the coat protein and there is a gene structure of double-coat protein, the exposed area of the coat protein is greatly increased and the affinity between the coat protein and the protein purification system is enhanced. After induction and expression, the protein purification tag is exposed on the surface of the coat protein, and the virus-like particle is captured by means of 6×His protein purification method, which may greatly simplify the complex operation procedure of preparing the virus-like particles while increase the purity of the virus-like particles.

(iii) Extremely high yield. In the case of a single-coat protein structure, the addition of the protein purification tag to the AB-loop hairpin structure may affect the packaging efficiency of the coat protein and reduce the yield of virus-like particles. The novel virus-like particle expression vector comprises a nucleotide sequence encoding the double-CP protein structure, which ensures the function of the protein purification tag added to the AB-loop hairpin structure without reducing the exogenous gene-packaging efficiency of the coat protein. Meanwhile, after mutation of the initiation codon of the maturase protein, the yield of the virus-like particles obtained finally may be increased effectively.

### BRIEF DESCRIPTION OF THE DRAWINGS

FIG. 1 shows the comparison of nucleic acid electrophoresis on lysate supernatants of the expressed products (after induction) of the recombinant strain containing the plasmid pTMSCA and the recombinant strain containing the plasmid pTrcHis-MS2 in Example 1 of the present invention. Wherein, lane 1 was the lysate supernatant (5 μL) of the expressed products of the recombinant strain containing the plasmid pTMSCA after induction; lane 2 was the lysate supernatant (5 μL) of the expressed products of the recombinant strain containing the plasmid pTrcHis-MS2 after induction; and M was DL2000 DNA Marker.

FIG. 2 shows the comparison of nucleic acid electrophoresis on lysate supernatants of the expressed products after packaging exogenous fragments of different length by the recombinant strain containing the plasmid pTMSCA and the recombinant strain containing the plasmid pTrcHis-MS2 in Example 1 of the present invention. Wherein, M is DL2000 DNA Marker; lane 1 was the lysate supernatant (5 μL) of the expressed products after packaging a exogenous gene fragment A (A<500 bp) by the recombinant strain containing the plasmid pTrcHis-MS2; lane 2 was the lysate supernatant (5 μL) of the expressed products after packaging a exogenous gene fragment B (1,000 bp>B >500 bp) by the recombinant strain containing the plasmid pTrcHis-MS2; lane 3 was the lysate supernatant (5 μL) of the expressed products after packaging a exogenous gene fragment C (1,500 bp>C>1,000 bp) by the recombinant strain containing the plasmid pTMSCA; and lane 4 was the lysate supernatant (5 μL) of the expressed products after packaging a exogenous gene fragment D (D>1,800 bp) by the recombinant strain containing the plasmid pTMSCA.

FIG. 3 shows the comparison of nucleic acid electrophoresis on lysate supernatants of the expressed products after packaging exogenous fragments of different length by the novel virus-like particle expression vector pTMSCA2C, the recombinant strain containing the plasmid pTMSCA and the vector pTrcMS in Example 2 of the present invention. Wherein, M was 1 kb DNA Marker; lane 1 was the lysate supernatant (5 μL) of the expressed products after packaging a exogenous gene fragment D (D>1,800 bp) by pTMSCA; lane 2 was the lysate supernatant (5 μL) of the expressed products after packaging a exogenous gene fragment A (A<500 bp) by pTrcMS; lane 3 was the lysate supernatant (5 μL) of the expressed products after packaging a exogenous gene fragment D (D>1,800 bp) by pTMSCA2C.

FIG. 4 shows the results of SDS-PAGE electrophoresis of the purified virus-like particle pTMSCA2C-SBV in Example 3. Wherein, lane 1 was the pTMSCA2C-SBV virus-like particle (Coomassie blue staining), and M was Spectra™ Multicolor Low Range Protein Ladder.

FIG. 5 is a fluorescence image of real time PCR detection of the purified virus-like particle pTMSCA2C-SBV in Example 3 of the present invention for the purpose of determining whether or not DNA residue is present.

FIG. 6 is an electron microscopy image of the virus-like particle pTMSCA2C-SBV in Example 3 of the present invention (100,000×JEM1400).

### BEST MODE

Without limitation on the scope of the present invention, the following Examples are for the purpose of explaining the present invention. Unless specified otherwise, the Examples are performed under conventional experimental conditions such as those recited in Molecular Cloning: A Laboratory Manual (Sambrook J & Russell D W, 2001) or under conditions suggested in the manufacturer's instructions.

### EXAMPLE 1

### Construction of a Recombinant Strain Containing a Plasmid pTMSCA

Firstly, using the plasmid pTrcHis-MS2 (SEQ ID NO: 1) as a template, the base T at position 5 of the gene sequence of MS2 bacteriophage 19mer packaging site was mutated into C by In-Fusion technology to give pTMSC; then, valine which was a amino acid corresponding to the initiation codon for encoding the maturase protein of MS2 bacteriophage was mutated into methionine to give pTMSCA.

The specific construction method is described as follows:

\textcircled{1} The base mutation was introduced into the PCR primers; using the plasmid pTrcHis-MS2 as a template, PCR amplification was performed by Primer” STAR HS DNA Polymerase with the Primer A primer pair and the Primer B primer pair respectively to give amplification product named PCR product A and PCR product B, wherein, the PCR amplification primers were shown in SEQ ID NOs: 4-7:

Primer A-F
5′-GAGGAATAAACCATGCGAGCTTTTAGTACCCTTG-3′;
Primer A-R
5′-TGGGTGATCCTCATGTTTGAATGGCCGGCGTC-3′;
Primer B-F
5′-GCCATTCAAACATGAGGATCACCCATGTCGAAG-3′;
and
Primer B-R
5′-GTTCGGGCCCAAGCTTCGAATTCCC-3′.

50 μL PCR amplification reaction system is as follows: 5×Prime STAR Buffer (Mg²⁺ plus), 10 μL; Prime® STAR HS DNA polymerase, 1.3 U; dNTP Mixture (each 2.5 mM), 4 μL; Primer A-F/R (20 pmol/μL), each 0.5 μL; Primer B-F/R (20 pmol/μL), each 0.5 μL; pTrcHis-MS2 DNA template, 10 ng; add ddH₂O to 50 μL. PCR amplification program: 30 cycles of denaturation at 98° C. for 10 s, annealing at 55° C. for 10 s, extension at 72° C. for 30 s; and extension at 72° C. for 10 min. The amplification product was recovered and purified.

\textcircled{2} The plasmid pTrcHis-MS2 was subjected to double restriction enzyme digestion with XhoI and HindIII. The digestion system is as follows: XhoIIHindIII, 10 U; 10×restriction enzyme digestion buffer, 5 μL; plasmid template, 1 μg; add ddH₂O to 10 μL. The reaction was performed at 37° C. for 2 hours. The product of the restriction enzyme digestion was subjected to gel recovery and purification respectively, and named as pTrcHis-MS2 (X/H).

\textcircled{3} Using In-Fusion HD Cloning Kit (Clontech Code No. 639648), PCR product A, PCR product B, and pTrcHis-MS (X/H) were linked together. The reaction system and conditions are as follows: PCR product A/B, each 200 ng; pTrcHis-MS2 (X/H), 100 ng; 5×In-Fusion HD Enzyme Premix 2 μL, add ddH₂O to 10 μL; the reaction was performed at 50° C. for 15 minutes.

\textcircled{4} 2.5 μL of the above-mentioned In-Fusion product was taken and transformed into E. coli Competent Cell JM109 through heat-shock transformation. The obtained product was cultured overnight at 37° C. The positive clones were picked and identified by sequencing. The primers used for identification were:

Primer-U1: 5′-GACAATTAATCATCCGGCTCG-3′, and Primer-L1: 5′-GATCTTCGTTTAGGGCAAGGTAG-3′ (SEQ ID NOs: 10-11). The plasmid identified as having the correct sequence was named pTMSC.

\textcircled{5} The mutated base was introduced into the PCR primers; using the plasmid pTMSC as a template, PCR amplification was performed by Primer STAR HS DNA Polymerase with the Primer 1 as a primer pair to give amplification product named PCR product I, wherein, the PCR amplification primers were shown in SEQ ID NOs: 8-9:

Primer1-F:
5′-GAGGAATAAACCATGCGAGCTTTTAGTACCCTTG-3′,
and
Primer1-R:
5′-CCACCTGCCGGCCACGTGTTTTGATC-3′.

50 μL PCR amplification reaction system is as follows: 5×Prime STAR Buffer (Mg²⁺ plus), 10 μL; Prime STAR HS DNA polymerase, 1.3 U; dNTP Mixture (each 2.5 mM), 4 μL; PrimerI-F/R (10 pmol/μL), each 0.5 μL; pTMSC DNA template, 10 ng; add ddH₂O to 50 μL. PCR amplification program: 30 cycles of denaturation at 98° C. for 10 s, annealing at 55° C. for 10 s, extension at 72° C. for 30 s; and extension at 72° C. for 10 min. The amplification product was recovered and purified.

\textcircled{6} The plasmid pTMSC was subjected to double restriction enzyme digestion with NcoI and PmaCI. The digestion system is as follows: NcoIIPmaCI, 10 U; 10×restriction enzyme digestion buffer, 5 μL; plasmid template, 1 μg; add ddH₂O to 10 μL. The reaction was performed at 37° C. for 2 hours. The product of the restriction enzyme digestion was subject to gel recovery and purification respectively, and named as pTMSC (N/P).

\textcircled{7} Using In-Fusion® HD Cloning Kit (Clontech Code No. 639648), the PCR product I and pTMSC (N/P) were linked together. The reaction system and conditions are as follows: PCR product I, 200 ng; pTMSC (N/P), 100 ng; 5×In-Fusion HD Enzyme Premix 2 μL, add ddH₂O to 10 μL; the reaction was performed at 50° C. for 15 minutes.

\textcircled{8} 2.5μL of the above-mentioned In-Fusion product was taken and transformed into E. coli Competent Cell JM109 through heat-shock transformation. The obtained product was cultured overnight at 37° C. The positive clones were picked and identified by sequencing. The primers used for identification were as follows:

Primer-U1: 5′-GACAATTAATCATCCGGCTCG-3′, and Primer-L1: 5′-GATCTTCGTTTAGGGCAAGGTAG-3′. The plasmid identified as having the correct sequence was named as pTMSCA.

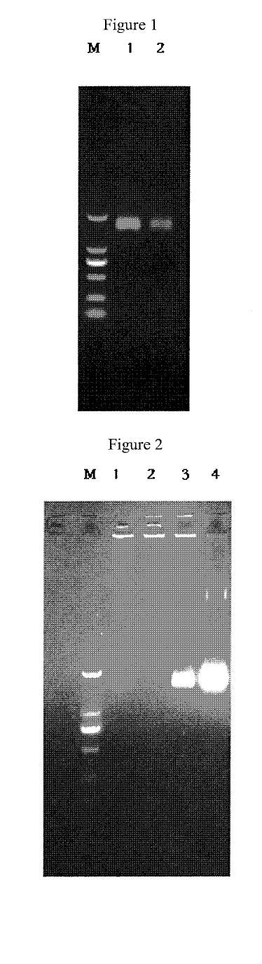

\textcircled{9} Comparison results between the lysate supernatants of pTMSCA and pTrcHis-MS2: the recombinant strains pTMSCA and pTrcHis-MS2 were respectively induced by IPTG (final concentration, 1 mol/L) to express for 16 hours, centrifugated at 5,000 rpm for 10 minutes to collect cells. The product precipitate was added with 20 μL 1×TE buffer (pH 8.0) per mL cell precipitate, mixed well by vortex, added with 1 μL lysozyme solution (25 mg/mL) per mL cell precipitate, digested at 37° C. for 30 minutes, and then centrifugated at 10,000 rpm for 10 minutes. Nucleic acid electrophoresis was performed on the supernatants obtained by pTMSCA and pTrcHis-MS2 and comparison between the yield of the expressed products was conducted (FIG. 1).

\textcircled{10} Comparison results between the lysate supernatants of the expressed products after exogenous fragments of different length packaged by pTMSCA and pTrcHis-MS2: the plasmid pTrcHis-MS2 was respectively linked with an exogenous gene fragment of different length (fragment A (A<500 bp) and fragment B (500 bp<B<1000 bp)) via two restriction enzyme digestion cutting sites for KpnI and HindIII, and then subjected to transformation and sequencing to construct recombinant plasmids pTrcHis-MS2-A and pTrcHis-MS2-B. Referring to the construction method of pTrcHis-MS2-A and pTrcHis-MS2-B, the recombinant plasmids pTMSCA-C (1000 bp<C<1500 bp) and pTMSCA-D (D>1800 bp) were constructed respectively 4 recombinant strains were processed referring to step \textcircled{9}, and comparison between the yield of the expressed products was conducted (FIG. 2).

### EXAMPLE 2

### Construction of a Novel Virus-Like Particle Expression Vector pTMSCA2C

Using the plasmid pTMSCA as a template, the synthesized double-CP gene encoding MS2 bacteriophage coat protein in which two CP genes were linked in series was inserted between XhoI/HindIII restriction cutting sites by the method of restriction enzyme digestion and insertion to give the novel virus-like particle expression vector pTMSCA2C, wherein, the first CP gene sequence was the gene sequence coding wild type MS2bacteriophage coat protein with the deletion of the stop codon thereof; and the second CP gene sequence was the gene sequence coding coat protein on the vector pTrcMS (SEQ ID NO: 2), which constituted a series structure together with the first CP gene sequence. The specific construction method is as follows:

\textcircled{1} Using the plasmid pTMSCA as a starting vector, the gene coding sequences corresponding to the MS2 bacteriophage coat protein were modified according to the following mode: the first CP gene sequence was the gene sequence coding wild type MS2 bacteriophage coat protein with the deletion of the stop codon thereof was deleted; the second CP gene sequence was the gene sequence coding coat protein on the vector pTreMS; the 2CP gene sequence synthesized by Huada Gene Biology Co., Ltd. was inserted into the vector pUC57; and the obtained recombinant vector was named as pUC57-2CP.

\textcircled{2} The plasmids pUC57-2CP and pTMSCA were subjected to double restriction enzyme digestion with XhoI and HindIII respectively. The digestion system is as follows: XhoI/HindIII, 10 U; 10×restriction enzyme digestion buffer, 5 μL; plasmid template, 1 μg; add ddH₂O to 10 μL. The reaction was performed at 37° C. for 2 hours. Respectively, the products of the restriction enzyme digestion were subjected to gel recovery and purification and named as pUC57 -2CP (X/H) and pTMSCA (X/H).

\textcircled{3} Using DNA ligation kit (Takara, Code No. 6022), the pUC57-2CP (X/H) and pTMSCA (X/H) were linked together. The reaction system and condition are as follows: pUC57-2CP (X/H)/pTMSCA (X/H), each 100 ng; solution I, 5 μL, add ddH₂O to 10 μL. The ligation reaction was performed overnight at 16° C.

\textcircled{4} 2.5 μL of the above-mentioned ligation product was taken and transformed into E. coli Competent Cell JM109 through heat-shock transformation. The obtained product was cultured overnight at 37° C. The positive clones were picked and identified by sequencing. The primers used for identification were: Primer-U1: 5′-GACAATTAATCATCCGGCTCG-3′ and Primer-L1: 5′-GATCTTCGTTTAGGGCAAGGTAG-3′.

The plasmid identified as having the correct sequence was named as pTMSCA2C.

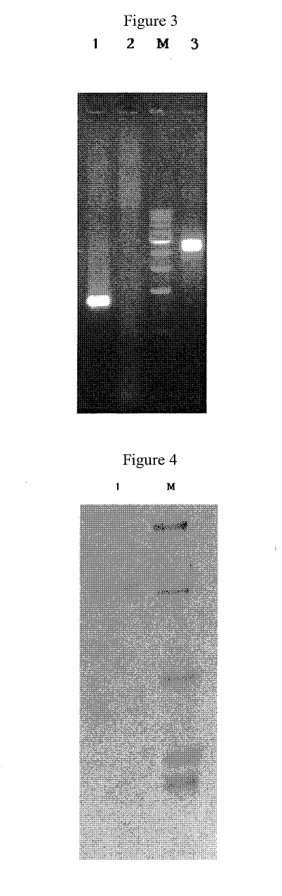

\textcircled{5} Comparison results between lysate supernatants of the expressed products after exogenous fragments of different length packaged by pTMSCA2C, pTMSCA and pTrcMS (ZL201110445022.5): referring to step ED in Example 1, pTMSCA2C-D (D>1,800 bp), pTMSCA-D (D>1,800 bp) and pTrcMS-A (A<500bp) were constructed by using plasmids pTMSCA2C, pTMSC-AP and pTrcMS. Three recombinant strains were processed referring to step \textcircled{9} in Example 1, and comparison between the yield of the expressed products was conducted (FIG. 3).

### EXAMPLE 3

### Preparation of Schmallenberg (SB) Virus-Like Particles

1. Construction of Plasmid pTMSCA2C-SBV

The plasmid pGEM-T-SBV and plasmid pTMSCA2C prepared in Example 2 were subjected to double restriction enzyme digestion with KpnI and HindIII and gel recovery and purification, respectively; and then linkage and sequencing were performed to construct the recombinant plasmid pTMSCA2C-SBV.

2. Preparation of pTMSCA2C-SBV Virus-Like Particles

\textcircled{1} Expression and lysis: Specific method was carried out referring to step \textcircled{9} in Example 1.

\textcircled{2} Using Ni Sepharose 6 Fast Flow purification system (GE) the product was recovered from the supernatant of pTMSCA2C-SBV and the collected filtrate was the purified virus-like particle pTMSCA2C-SBV. The product was identified by SDS-PAGE protein electrophoresis.

\textcircled{3} RNA was extracted from the recovered virus-like particles and subjected to real-time PCR and RT-PCR to identify the purity of the obtained RNA solution. The primers used for identification were Primer-SBV-F: 5′-TCAGATTGTCATGCCCCTTGC-3′ and Primer-SBV-R: 5′-TTCGGCCCCAGGTGCAAATC-3′, respectively. Probe: 5′-FAM-TTAAGGGATGCACCTGGGCCGATGGT-3′. See SEQ ID NOs: 12-14.

3. Morphological Identification of pTMSCA2C-SBV Virus-Like Particles

Firstly, the purified solution of virus-like particles was subjected to 1% uranyl acetate staining, then subjected to natural drying and finally subjected to morphological observation through a transmission electron microscope.

4. Clinical Application of the pTMSCA2C- SBV Virus-Like Particles

Using purified SBV virus-like particle as a positive quality control product, SBV nucleic acid testing was performed on sheep serum clinical samples.

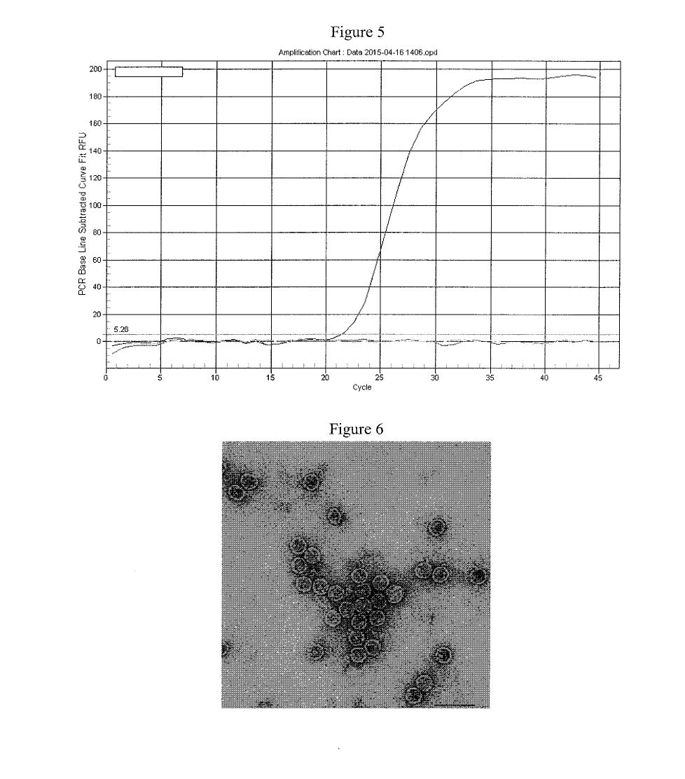

The purified SBV virus-like particles were detected by SDS-PAGE electrophoresis. Results were shown in FIG. 4. The target protein was located between 26 kDa and 48 kDa, the size of which was consistent with a size of 2 times of MS2 bacteriophage coat protein (27.4 kDa). Verification results of purity indicated that: for PCR identification, there was no amplification curve; for RT-PCR identification, there was a standard S amplification curve, suggesting that there were virus-like particles containing the SB target gene in the solution without DNA contamination (FIG. 5). Electron microscopy observation showed that polygonal particles with a diameter of about 26 nm, i.e. the expressed virus-like particles after induction, may be observed (FIG. 6). Clinical application tests indicated that after fluorescence quantitative detection, the nucleic acid detection of the positive quality control product of SBV virus-like particles showed a standard S curve, and the clinical serum samples were identified as negative by the nucleic acid detection, which was consistent with the serologic detection results.

Although the present invention is described in detail through the general description and specific embodiments above, modifications or improvements may be made based on the present invention, which is obvious to a person skilled in the art. Therefore, all of those modifications or improvements that are made without departing from the spirit of the present invention fall into the scope of the invention as claimed.

### INDUSTRIAL APPLICABILITY

The present invention discloses a novel virus-like particle expression vector and a construction method thereof. When the virus-like particle is prepared by using the expression vector of the present invention, the yield and purity of the virus-like particle may be improved while the workload for preparation of virus-like particles may be greatly reduced.

### REFERENCE DOCUMENTS

[1] Pasloske B L, et al Armored RNA technology for production of ribonuclease-resistant viral RNA controls and standards. J Clin Microbiol, 1998, 36(12):3590-3594;Walker Peach C R, et al. Ribonuclease resistant RNA controls (Armored RNA) for reverse transcription-PCR, branched DNA, and genotyping assays for hepatitis C virus. Clin Chem,1999, 45(12):2079-2085.

[2] Talbot S J, Goodman S, Bates S R, et al. Use of synthetic oligoribonucleotides to probe RNA-protein interactions in the MS2 translational operator complex. Nucleic Acids RES, 1990, 18(12):3521-3528.

[3] Qiuying E I, Yangjian C, Qiwei G, et al. Preparation of a chimeric Armored RNA as a versatile calibrator for multiple virus assays. Clin Chem,2006, 52(7):1446-1448.

[4] Yuxiang Wei, Changmei Yang, Baojun Wei, et al. RNase-Resistant virus-like particles containing long chimeric RNA sequences produced by two-plasmid coexpression system. Journal of clinical microbiology,2008, 46(5):1734-1740.

1. A novel virus-like particle expression vector pTMSCA2C, characterized in that the expression vector pTMSCA2C is constructed as follows: using plasmid pTrcHis-MS2 as a starting vector and mutating the base T at position 5 of the gene sequence of MS2 bacteriophage 19mer packaging site on the plasmid pTrcHis-MS2 into C through genetic mutation technologies to obtain a plasmid pTMSC; then mutating valine which is an amino acid corresponding to the initiation codon on the plasmid pTMSC for encoding the maturase protein of MS2 bacteriophage into methionine to obtain a plasmid pTMSCA; and finally, the gene sequence coding wild type MS2 bacteriophage coat protein, after the removal of the terminator, is linked in series with the gene sequence coding MS2 bacteriophage coat protein comprising histidine-tag which is from a pseudovirus vector pTrcMS, and the gene sequence obtained after linking in series is linked to the plasmid pTMSCA to give pTMSCA2C;
wherein, the nucleotide sequence of the plasmid pTrcHis-MS2 is set forth in SEQ ID NO: 1, the nucleotide sequence of the pseudovirus vector pTrcMS is set forth in SEQ ID NO: 2 and the nucleotide sequence of the expression vector pTMSCA2C is set forth in SEQ ID NO: 3.

2. (canceled)

3. A method for constructing the expression vector of claim 1, characterized in that the method comprises the following steps:
1) preparation of the plasmid pTMSC: using the plasmid pTrcHis-MS2 as a template, PCR amplification is performed with Primer A and Primer B into which a base mutation is introduced respectively to obtain PCR product A and PCR product B, and then the amplified products are recovered and purified; the plasmid pTrcHis-MS2 is subjected to double restriction enzyme digestion with XhoI and HindIII and the digested plasmid pTrcHis-MS2 is linked with the PCR product A and PCR product B by In-Fusion technique and transformed into a recipient; and after screening and identification, the plasmid pTMSC is obtained;
2) preparation of the plasmid pTMSCA: using the plasmid pTMSC as a template, PCR amplification is performed with Primer 1 into which a base mutation is introduced to obtain PCR product I, and then the amplified product is recovered and purified; the plasmid pTMSC is subjected to double restriction enzyme digestion with NcoI and PmaCI and the digested plasmid pTMSC is linked with the PCR product I by In-Fusion technique and transformed into a recipient; and after screening and identification, the plasmid pTMSCA is obtained; and
3) construction of the expression vector pTMSCA2C: a gene sequence coding wild type MS2 bacteriophage coat protein, after the removal of terminator, is linked in series with a gene sequence coding MS2 bacteriophage coat protein comprising histidine-tag from the pseudovirus vector pTrcMS, and the gene sequence obtained after linking in series is inserted into the plasmid pTMSCA between XhoI and HindIII restriction enzyme cutting sites to give the expression vector pTMSCA2C;
wherein, the sequences of the Primer A, Primer B and Primer 1 are as follows:
Primer A-F:
(SEQ ID NO 8)
5′-GAGGAATAAACCATGCGAGCTTTTAGTACCCTTG-3′
Primer A-R:
(SEQ ID NO 5)
5′-TGGGTGATCCTCATGTTTGAATGGCCGGCGTC-3′;
Primer B-F:
(SEQ ID NO 6)
5′-GCCATTCAAACATGAGGATCACCCATGTCGAAG-3′;
Primer B-R:
(SEQ ID NO 7)
5′-GTTCGGGCCCAAGCTTCGAATTCCC-3′;
Primer 1-F:
(SEQ ID NO 8)
5′-GAGGAATAAACCATGCGAGCTTTTAGTACCCTTG-3′;
and
Primer 1-R:
(SEQ ID NO 9)
5′-CCACCTGCCGGCCACGTGTTTTGATC-3′.

4. The method of claim 3, characterized in that, the primers used for identification in steps 1) and 2) are:
Primer-U1:
(SEQ ID NO 10)
5′-GACAATTAATCATCCGGCTCG-3′,
and
Primer-L1:
(SEQ ID NO 11)
5′-GATCTTCGTTTAGGGCAAGGTAG-3′.

5. A virus-like particle, characterized in that the virus-like particle comprises a RNA transcript of an exogenous gene carried by the novel virus-like particle expression vector pTMSCA2C of claim 1.

6. A method for the preparation of virus-like particles, characterized in that, the method comprises the following steps: an exogenous gene fragment is cloned into the downstream of the gene sequences coding MS2 bacteriophage coat protein linked in series in the novel virus-like particle expression vector pTMSCA2C of claim 1; then, a terminator is inserted into the downstream of the exogenous gene fragment; after transcription, RNA transcripts of the exogenous gene carrying a RNA sequence of a bacteriophage operator are obtained; bacteriophage coat proteins are expressed after induction and assembled into protein coats while the RNA transcripts of the carried exogenous gene are encapsulated into the protein coats to give the virus-like particles.

7. (canceled)

8. A quality control product prepared from the virus-like particle of claim 5, used for the detection of pathogenic microorganisms.

9. A method for the preparation of a quality control product for the detection of pathogenic microorganisms which comprises using the virus-like particle expression vector of claim 1.

10. A method for the preparation of a quality control product for the detection of pathogenic microorganisms which comprises using the virus-like particle expression vector of claim 5.

11. A quality control product prepared from the virus-like particle of claim 1, used for the detection of pathogenic microorganisms.


SEQUENCE LISTING (14 sequences)

SEQ ID NO: 1 - DNA, 6101 bp, Artificial sequence
  Plasmid pTrcHis-MS2 synthesized from partial sequence of expression vector pTrcHis2A and coding sequence of mature enzyme protein and coat protein of phage MS2
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgcctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtggctgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtcgaagacaacaaagaagttcaatctagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 2 - DNA, 6119 bp, Artificial sequence
  Pseudovirus vector pTrcMS synthesized based on sequence of Plasmid pTrcHis-MS2
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggacatcaccatcaccatcacactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggattacccatgtcgaagacaacaaagaagttcaatctagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 3 - DNA, 6316 bp, Artificial sequence
  Virus-like particle expression vector pTMSCA2C synthesized based on Plasmid pTrcHis-MS2
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgcctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtggctgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctaccatggcttctaactttactcagttcgttctcgtcgacaatggcggacatcaccatcaccatcaccatcacactggcgacgtggctgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccattcaaacatgaggatcacccatgtcgaagacaacaaagaagttcaatctagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtg

SEQ ID NO: 4 - DNA, 34 bp, Artificial sequence
  synthesized
gaggaataaaccatgcgagcttttagtacccttg

SEQ ID NO: 5 - DNA, 32 bp, Artificial sequence
  synthesized
tgggtgatcctcatgtttgaatggccggcgtc

SEQ ID NO: 6 - DNA, 33 bp, Artificial sequence
  synthesized
gccattcaaacatgaggatcacccatgtcgaag

SEQ ID NO: 7 - DNA, 25 bp, Artificial sequence
  synthesized
gttcgggcccaagcttcgaattccc

SEQ ID NO: 8 - DNA, 34 bp, Artificial sequence; identical to SEQ ID NO: 4
  synthesized

SEQ ID NO: 9 - DNA, 26 bp, Artificial sequence
  synthesized
ccacctgccggccacgtgttttgatc

SEQ ID NO: 10 - DNA, 21 bp, Artificial sequence
  synthesized
gacaattaatcatccggctcg

SEQ ID NO: 11 - DNA, 23 bp, Artificial sequence
  synthesized
gatcttcgtttagggcaaggtag

SEQ ID NO: 12 - DNA, 21 bp, Artificial sequence
  synthesized
tcagattgtcatgccccttgc

SEQ ID NO: 13 - DNA, 20 bp, Artificial sequence
  synthesized
ttcggccccaggtgcaaatc

SEQ ID NO: 14 - DNA, 26 bp, Artificial sequence
  synthesized
ttaagggatgcacctgggccgatggt